(19) United States
(12) Patent Application Publication
(10) Pub. No.: US 2018/0273598 A1
(43) Pub. Date: Sep. 27, 2018

(54) ADHD MARKER, APPLICATION THEREOF AND KIT

(71) Applicants: WENZHOU KANGNING HOSPITAL CO., LTD., Wenzhou (CN); INSTITUTE OF HYDROBIOLOGY, CHINESE ACADEMY OF SCIENCES, Wuhan (CN)

(72) Inventors: Xi LI, Wenzhou (CN); Zhan YIN, Wuhan (CN); Kezhi DAI, Wenzhou (CN); Minjie YE, Wenzhou (CN); Xuyan PENG, Wuhan (CN); Xianyong ZHOU, Wenzhou (CN); Miaomiao ZHENG, Wenzhou (CN)

(73) Assignees: WENZHOU KANGNING HOSPITAL CO., LTD., Wenzhou (CN); INSTITUTE OF HYDROBIOLOGY, CHINESE ACADEMY OF SCIENCES, Wuhan (CN)

(21) Appl. No.: 15/730,473

(22) Filed: Oct. 11, 2017

(30) Foreign Application Priority Data
Mar. 22, 2017 (CN) 201710173706.1

(51) Int. Cl. C07K 14/575; G01N 33/74; A61K 31/185

(52) U.S. Cl. CPC C07K 14/575; G01N 33/74; G01N 2800/305; G01N 2333/575; A61K 31/185

(57) ABSTRACT

The present invention provides a marker for ADHD and applications thereof. The marker is ghrelin, and the marker can screen for potential ADHD patients by detecting the marker in peripheral blood or cerebrospinal fluid. Further, the present invention provides a new drug for the treatment of ADHD, providing the possibility and hope of new treatment for ADHD patients.

### CROSS-REFERENCE TO RELATED APPLICATIONS

This Non-provisional application claims priority under 35 U.S.C. § 119(a) on Patent Application No(s). 201710173706.1 filed in People's Republic of China on Mar. 22, 2017, the entire contents of which are hereby incorporated by reference.

### BACKGROUND OF THE INVENTION

### Field of the Invention

This invention pertains to the field of medicine and particularly relates to an ADHD marker, application thereof and a kit.

### Description of the Related Art

ADHD, also known as Attention Deficit Hyperactivity Disorder (ADHD), is a common kind of childhood behavior disorders. According to foreign reports, the prevalence of ADHD is between 5% to 10%, and more than 10% according to domestic survey. These children's intelligence is normal or essentially normal, but they have defects in learning, behavior and emotional aspects, mainly for the inattention, short attention span, hyperactivity, emotional impulsivity, poor academic performance. If they can not get proper treatment, some children still have symptoms after adulthood, which significantly affect the patient's study, physical and mental health, adult family life and social skills.

Clinical drugs can be used to improve attention deficit, lower activity levels, improve academic performance to a certain extent and relations with family members of patients in the short term. Methylphenidate, a drug widely used in clinical currently, helps improve attention, high doses can improve hyperactivity, impulsivity symptoms and reduce behavioral problems. But its side effects, such as loss of appetite, insomnia, headache, restlessness and irritability, are obvious. Tomoxetine, which is listed as the first-line treatment drug for ADHD, has fewer adverse reactions, but its slow onset time is not suitable for the treatment of patients with acute ADHD. Therefore, it is extremely important to explore new therapeutic drugs and drug targets.

### BRIEF SUMMARY OF THE INVENTION

To overcome at least one deficiency of the prior art, this invention provides a novel biomarker for ADHD and a drug for the treatment of ADHD in children.

To achieve the above object, the invention adopts the following technical scheme:

The invention provides a marker for ADHD, and the marker is ghrelin. The human ghrelin is an endogenous neuropeptide composed of 28 amino acids and produced by gastrointestinal tract, having a variety of physiological functions including stimulating appetite.

Further, the potential ADHD patients can be screened by detecting an expression level of the ghrelin in collected human peripheral blood or cerebrospinal fluid. The potential ADHD patients can be screened accordingly, if a reduction of the expression of the ghrelin is detected.

The invention also provides an application of the aforementioned ADHD marker in the preparation of a reagent for detecting ADHD.

The invention also provides an application of a receptor GHSR-1a of the aforementioned ADHD marker in the preparation of a drug/medicament for the treatment of ADHD, and a receptor of the ghrelin is used as a drug target for the treatment of ADHD.

The invention further provides an application of a receptor agonist of the aforementioned ADHD marker in the preparation of a drug/medicament for the treatment of ADHD. The receptor agonists of the gherlin in the invention includes: alexamorelin, anamorelin, anamorelin fumarate, anamorelin hydrochloride, mesylate, ibutamoren mesylate, GHRP, ghrelin, acylated ghrelin and so on.

Further, the receptor agonist is methanesulfonic acid or mesylate.

The invention also provides a kit for detecting ADHD, and the kit includes a reagent for detecting a level of the ghrelin in human peripheral blood or cerebrospinal fluid. By detecting changes in the expression levels of ghrelin and acylated ghrelin in the individual, whether the person is a potential ADHD patient can be detected.

Compared with the prior art, the invention has the following advantages:

The invention provides a novel marker, a drug targets and drug treatment for ADHD, the marker can be detected in peripheral blood or cerebrospinal fluid to screen potential patients with ADHD. Further, the invention provides a new drug for the treatment of ADHD, providing the possibility and hope of new treatment of ADHD patients.

In order to make the above and other objects, features and advantages of the present invention more obvious and easy to understand, the following description of the preferred embodiment will be described in detail with accompanying drawings.

### BRIEF DESCRIPTION OF THE DRAWINGS

FIG. 1A shows a binding site for TALEN in Embodiment One;

FIG. 1B shows an identification of zebrafish genotype in Embodiment One;

FIG. 1C shows PCR product sequencing results;

FIG. 2A shows the comparison of movement distances of adult male zebrafish;

FIG. 2B shows the comparison of movement distances of larvae zebrafish;

FIG. 3A shows the movement of the adult zebrafish under light stimulation;

FIG. 3B shows the comparison of the activities of the adult zebrafish under light stimulation;

FIG. 4 shows the results of wild-type larvae zebrafish treated with YIL781;

FIG. 5 shows the comparison of ghrelin mutant zebrafish after mesylate treatment; and

FIG. 6 shows the results of ghrelin mutant zebrafish after methylphenidate treatment.

### DETAILED DESCRIPTION OF THE INVENTION

### Embodiment 1

Identification of Ghrelin Mutants in Zebrafish.

1) Generation of Ghrelin Mutants by TALEN

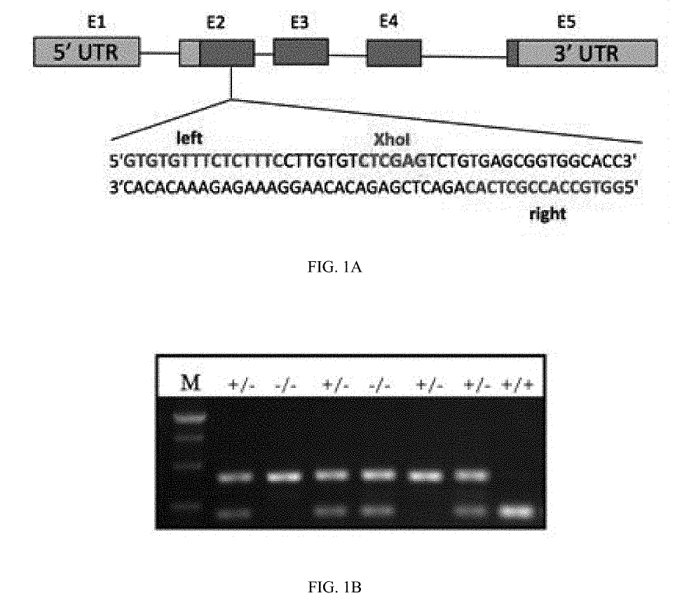

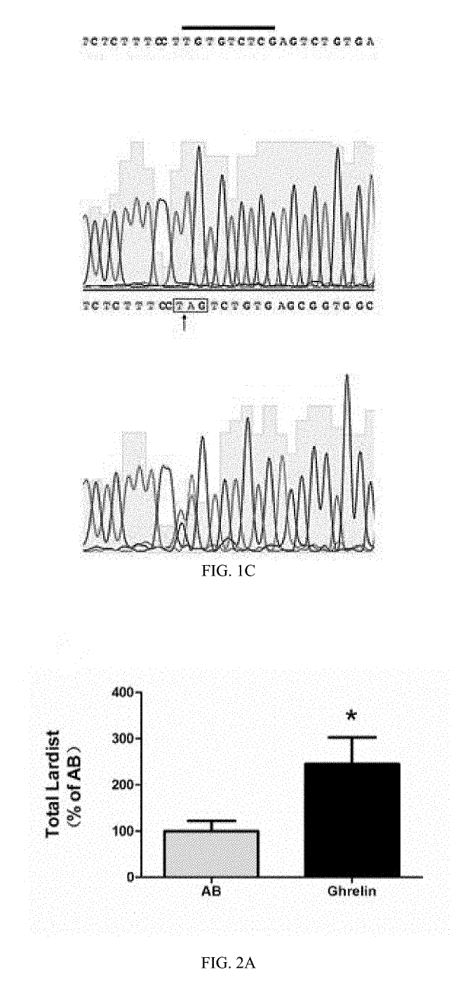

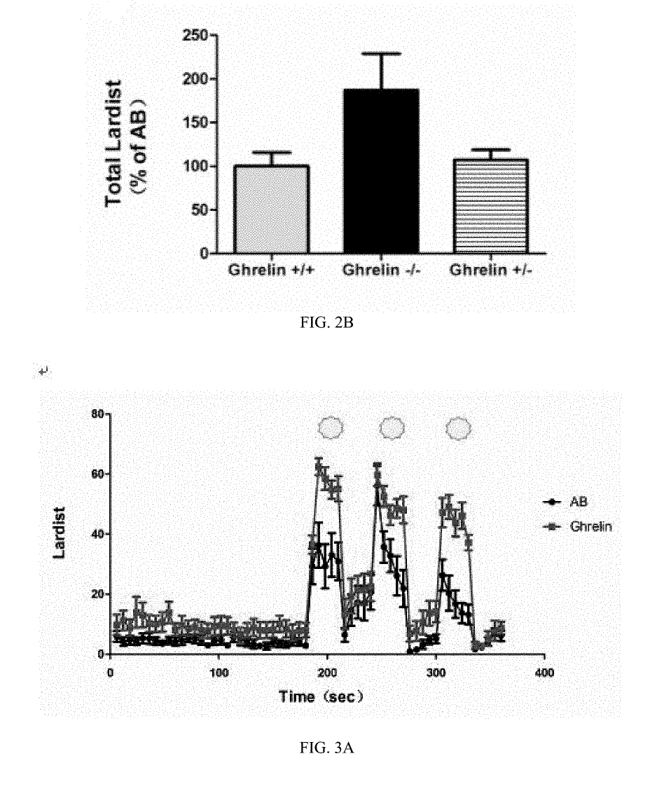

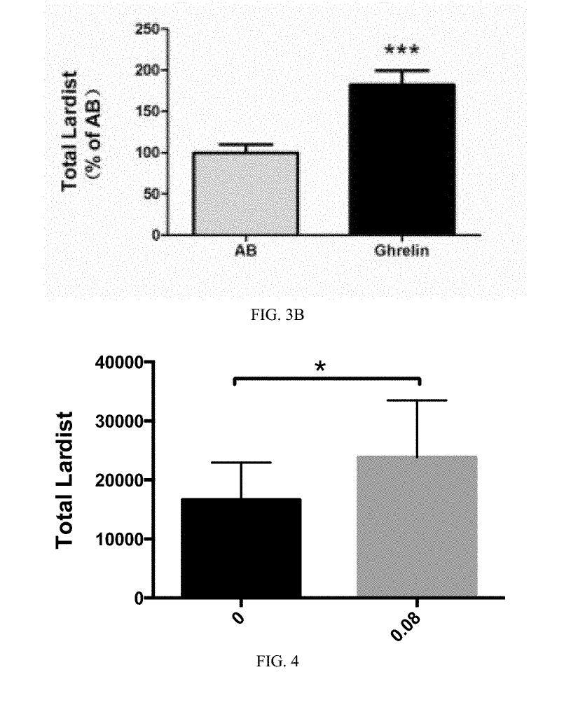

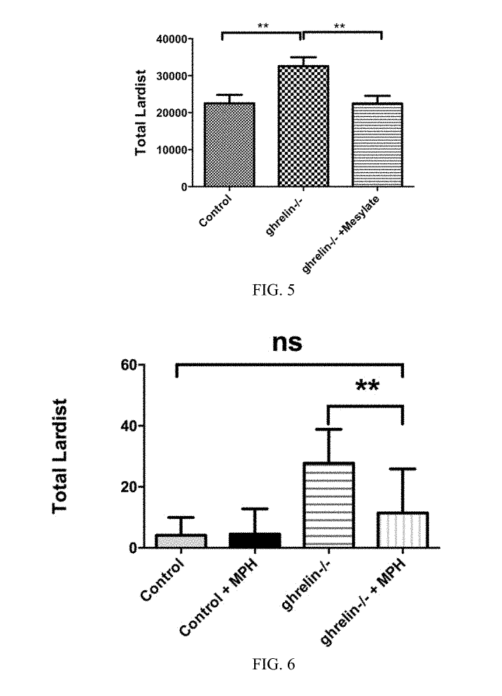

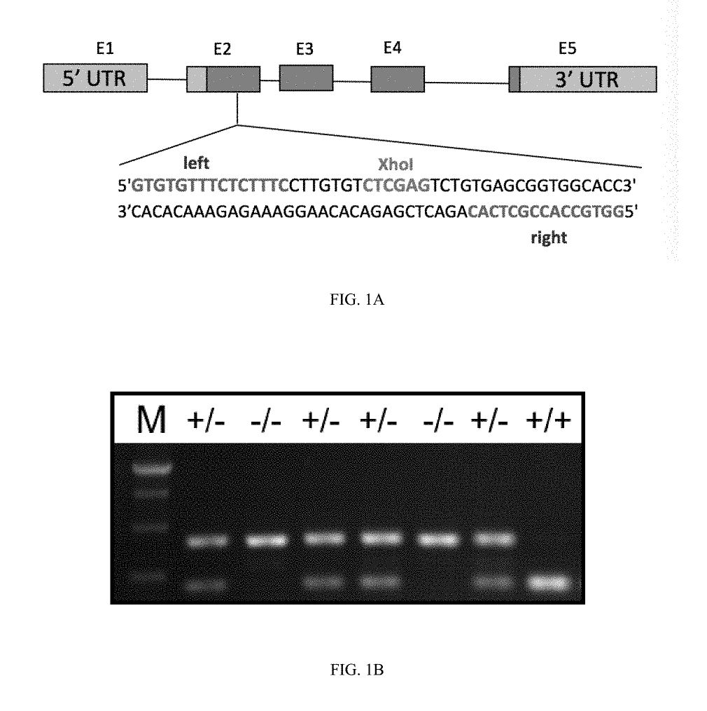

The zebrafish ghrelin mutant is artificially engineered and its nucleotide sequence encoding ghrelin is shown in SEQ NO.2. FIG. 1A shows the binding site for TALEN in this embodiment.

The zebrafish ghrelin nucleotide sequence is shown in SEQ NO.1, and the mutated nucleotide sequence is shown in SEQ NO.2. Compared with the SEQ NO. 1, nine nucleotides (TGTGTCTCG) was deleted in the ghrelin mutant and a termination code was generated which identified by sequencing which cannot synthesize the complete ghrelin precursor polypeptide and only encoded MPLRCRASSMFLLLCVSLS*. The expression of the ghrelin precursor polypeptide is predicted to be reduced in mutated individuals. The study shows that the mutant individual shows symptoms of ADHD-like symptoms. Thus, ghrelin and ghrelin precursor polypeptide can be used as a marker of ADHD.

As a result of the damage of the Xho1 restriction enzyme site after gene deletion, the mutated sequence cannot be cleaved by Xho1.

2) Genotyping

Primers were used as followed for genotyping, primer F: AGACC TACTG AGGCA GCCTC ATCA, Primer R: CCGAT CGTCT TCTTT GATCA CTGG. The template genome was prepared from the Genomic Extraction Kit (provided by Shanghai Generay Bio). All the PCR reagents were supplied by Qingdao Takara Company. Samples were added according to the following reaction system:

Ghrelin PCR System

| | ul | |
|---|---|---|
| 10X bufer | 2 | |
| dNTPmix | 0.5 | |
| primer F | 0.2 | |
| Primer R | 0.2 | |
| Template | 1 | |
| Taq | 0.25 | |
| dd water | 15.85 | |
| Total | 20 | μL |

| | |
|---|---|
| 95° C. 1 min | STAGE 1 |
| 95° C. 15 S | STAGE 2 |
| 58° C. 30 S X40 | |
| 72° C. 30 S | |
| 72° C. 7 min | STAGE 3 |
| 4° C. ∞ | STAGE 4 |

PCR products were confirmed by agarose gel electrophoresis, and part of the products was sequenced by Nanjing genscript biological company. Part of the PCR products was further digested with Xho1.

The reaction system is as following:

| | ul | |
|---|---|---|
| Template | 15 | |
| Buffer R | 2 | |
| XhoI | 0.3 | |
| dd water | 2.7 | |
| Total | 20 | μL |

FIG. 1B shows an identification of zebrafish genotype in Embodiment 1. FIG. 1C shows the PCR product sequencing results. As shown in FIG. 1B, the PCR product was digested with Xho1 and subjected to 1.5% agarose gel electrophoresis. In FIG. 1C, the PCR product of ghrelin mutant had a 9 nucleotides deletion (TGTGTCTCG) in exon 2, resulting in a new stop codon, TAG.

### Embodiment 2

The Tracking of Zebrafish Behavior.

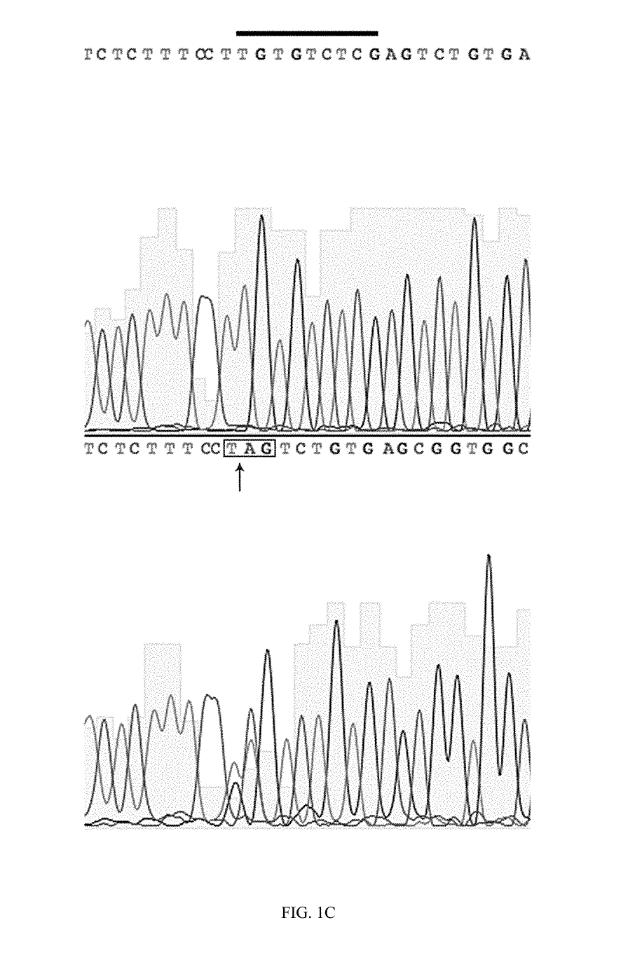

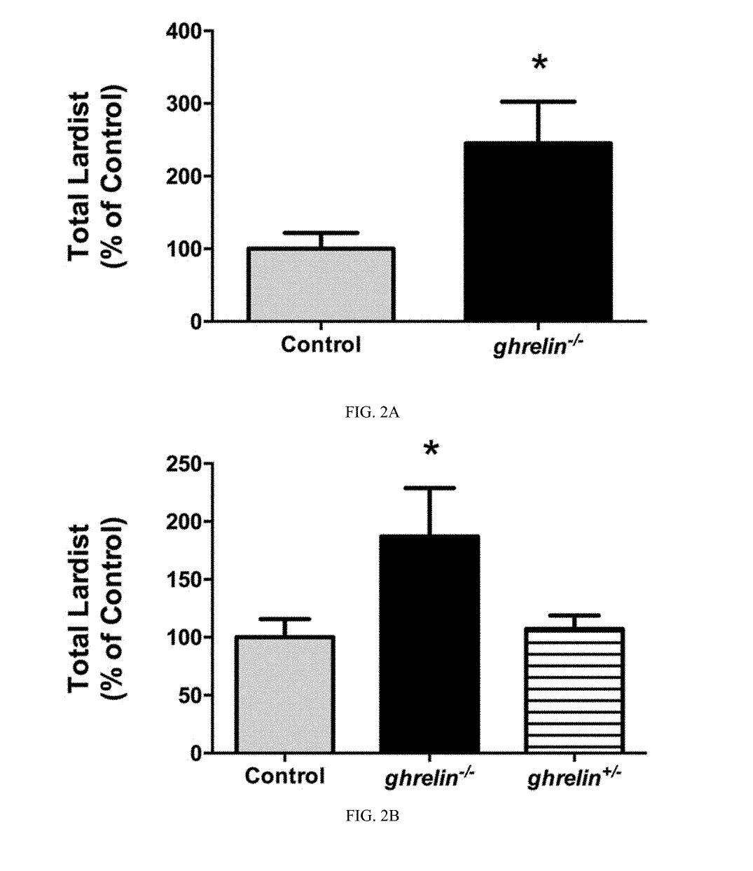

1) In order to detect whether ghrelin mutants had an ADHD-like phenotype, 3-8 months old adult zebrafish (control group) and mutants were placed in separate fish tanks, (ghrelin^{+/+} (AB or Control) and ghrelin^{-/-} (ghrelin mutant) were produced by ghrelin^{+/} intercross, then they were maintained to produce 3-8 months old adult zebrafish), the zebrafish activity was tracked using behavioral recorder viewpoint within 10 minutes, the ordinate represented total moving distance as shown in FIG. 2A.

The viewpoint behavioral recorder is used to track activity of the zebrafish. The parameter settings on the behavior recorder are shown in the table below:

The record parameters of adult zebrafish behavior

| | parameters |
|---|---|
| Standard distance | 12 cm |
| Total time | 10 min |
| Recording frequency | 10 s/time |
| Infrared parameters | Transparent 55 |
| Movement threshold | 3 cm/sec, 8 cm/sec |

FIG. 2A is a comparison of ghrelin mutant and control group of the adult male zebrafish. As shown in FIG. 2A, the movement distance of ghrelin mutant adult male zebrafish was significantly increased compared to control group. The ghrelin mutant showed ADHD-like hyperactivity phenotype.

2) 3 dpf (day post fertilization) of zebrafish embryos were produced by ghrelin^{+/-} (heterozygous) intercross. Pick a single hatched 3 dpf zebrafish larvae into a 96-well plate, the behavior is tested using following parameters. After test, the genotype of the larvae zebrafish was determined by PCR and enzyme digestion.

Larvae zebrafish behavior record parameters

| | parameters |
|---|---|
| Standard distance | 10 cm |
| Total time | 24 h |
| Recording frequency | 600 s/time |
| Infrared parameters | Transparent 10 |

FIG. 2B shows the comparison of ghrelin mutants, heterozygotes and control group in zebrafish larvae. As shown in FIG. 2B, the activity and movement distance of ghrelin mutant were increased by one fold compared to control zebrafish, while the ghrelin heterozygote had no significantly difference compared to the control group.

### Embodiment 3

The Tracking of Zebrafish Behavior with Light Stimulation.

3-8 month old adult zebrafish were placed in a separate fish tank, and the activity of the zebrafish was tracked by viewpoint behavioral recorder. The parameters setting on the behavior recorder is carried out as in Embodiment 1, and the intensity and time setting of the light stimulus were carried out according to the following table.

Behavior record parameters of zebrafish larvae

| | parameters |
|---|---|
| 180000 ms | 0% |
| 30000 ms | 100% |
| 30000 ms | 0% |
| 30000 ms | 100% |
| 30000 ms | 0% |
| 30000 ms | 100% |
| 30000 ms | 0% |

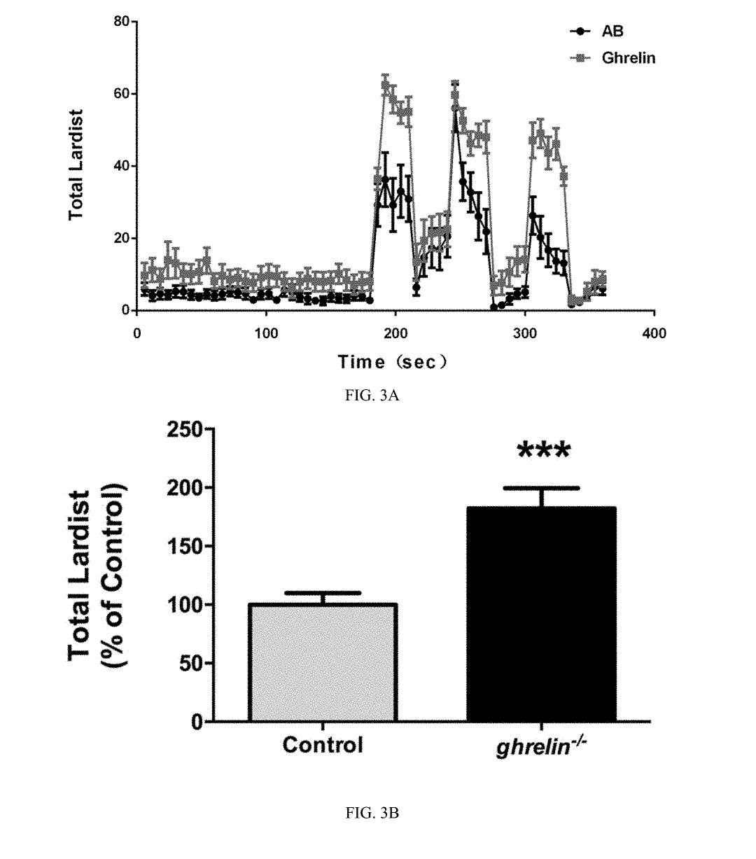

FIG. 3A shows the movement of the adult zebrafish under light stimulation. FIG. 3B shows the comparison of the activities of the adult zebrafish under light stimulation. As shown in FIGS. 3A and 3B, the movement distance of ghrelin mutant significantly increased in contrast to the control group under light stimulation.

### Embodiment 4

The Antagonists of Ghrelin Receptor GHSR-1a can Enhance Zebrafish Activity.

Zebrafish embryos (AB) were treated with different concentrations of YIL781 (0.08 uM, 0.4 uM, 2 uM, 10 uM, 50 uM) from 6 hpf to 3 dpf, and the behavior was detected by viewpoint behavioral recorder. The concrete scheme was as follows:

Behavior record parameters of zebrafish larvae

| | parameters |
|---|---|
| Standard distance | 10 cm |
| Total time | 15 h |
| Recording frequency | 600 s/time |
| Infrared parameters | Transparent 10 |

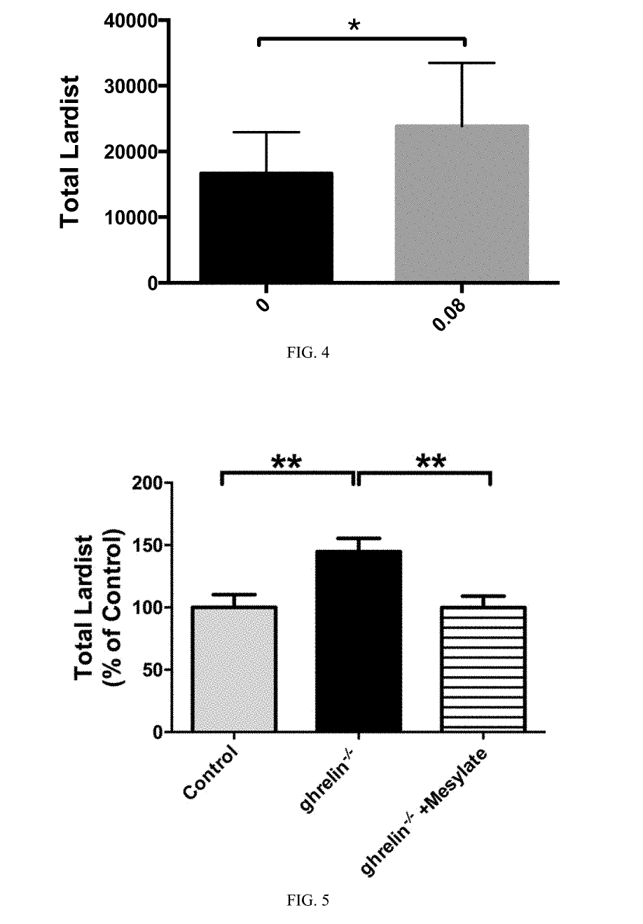

FIG. 4 shows the results of wild larvae zebrafish treated with concentrations of 0.08 uM/L YIL781. The results showed that YIL781 competitively inhibited the function of endogenous ghrelin and induced normal zebrafish to produce a hyperactivity phenotype.

### Embodiment 5

The Agonist of Ghrelin Receptor GHSR-1a can Rescue the ADHD-Like Phenotype in Ghrelin Mutant.

Embryos produced by ghrelin^{+/+}, ghrelin^{-/-} zebrafish intercross were treated with 10 uM Mesylate at 6 hpf. Select single hatched 3 dpf zebrafish larvae into a 96-well plate. The behavior was tested as shown in the following table. After test, the genotype of the zebrafish larvae was identified by PCR and enzyme digestion.

Behavior record parameters of zebrafish larvae

| | parameters |
|---|---|
| Standard distance | 10 cm |
| Total time | 24 h |
| Recording frequency | 600 s/time |
| Infrared parameters | Transparent 10 |

FIG. 5 is the comparison chart of Mesylate treated ghrelin mutant zebrafish.

As shown in the FIG. 5, ghrelin^{-/-} embryo was treated with 10 uM Mesylate at 6 hpf, and the movement distance was measured by behavior recorder at 3 dpf. Ghrelin mutant had a reduced movement distance after treatment with Mesylate and was not significantly different with the control group, that is, hyperactivity symptoms were rescued.

The invention has constructed ghrelin mutant zebrafish, and it has been found that adult zebrafish and larvae fish are hyperactive, and this phenotype can be rescued by mesylate, an agonist of ghrelin receptor GHSR-1a. At the same time, GHSR-1a blockers can induce ADHD like phenotype, similar to ghrelin mutants.

### Embodiment 6

Methylphenidate can be Used to Cure Hyperactivity Phenotypes in Ghrelin Mutant.

The embryos produced by ghrelin+/+, ghrelin−/− zebrafish intercross were treated with Methylphenidate at 72 hpf for 1 h. Pick a single hatched zebrafish larvae into a 96-well plate, behavior was tested as following table. After test, the genotype of the zebrafish larvae was identified by PCR and enzyme digestion.

Behavior record parameters of larvae zebrafish

| | parameters |
|---|---|
| Standard distance | 10 cm |
| Total time | 24 h |
| Recording frequency | 600 s/time |
| Infrared parameters | Transparent 10 |

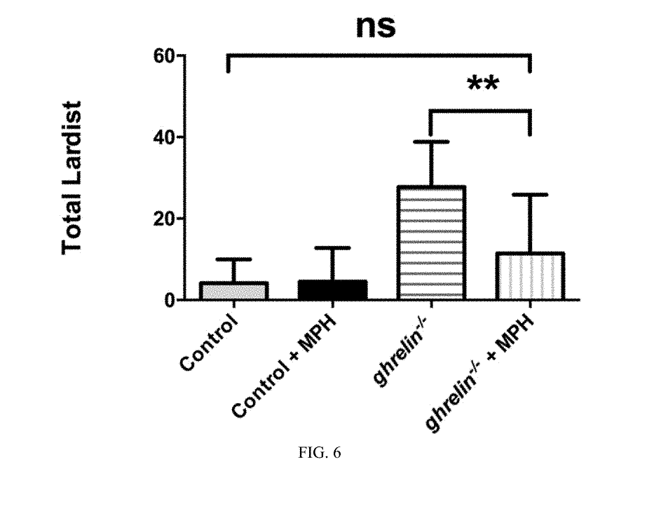

FIG. 6 shows the results of methylphenidate treatment of ghrelin mutant zebrafish.

The ghrelin^{-/-} embryos were treated with 10 uM methylphenidate for 1 h at 3 dpf, and the movement distance was measured by behavior recorder.

As shown in the FIG. 6, the ghrelin mutant zebrafish decreased its movement distance after treatment with methylphenidate and showed no significantly different with the control group. Therefore, methylphenidate, a drug of treatment of human ADHD, has a therapeutic effect on ghrelin mutant zebrafish.

### Embodiment 7

Detecting Total Ghrelin and Acylated Ghrelin in the Human Sample by ELISA.

### Experimental Procedure

Seeding Antibody

The antibody (50% glycerol, −20° C.) was diluted 1:4000 in 1×PBS, and 100 μL antibody was added to each well of a 96-well ELISA plate and sealed in a 4° C. refrigerator overnight.

Target (Standard) Protein Incubation

Pour out the substrate liquid in ELISA plate the next day, add 200 μL PBST to wash 3 times. The last time need 30 min to wash and then discard the PBST.

Each of plasma (100 ul) was added (diluted with 1×PBS according to require) to each well, sealed and incubated at 37° C. for 2 h.

Primary Antibody Incubation.

Incubate plasma for 2 hours, poured out the liquid in the wells, washed three times with PBST. 100 μL of rabbit anti-Ghrelin polyclonal antibody (biotin) at a dilution ratio of 1:4000 (diluted in PBST) was added to each well. Seal the plate and incubate it in a 37° C. incubator for 1 h.

Secondary Antibody Incubation

After 1 hour incubation of primary antibody, the liquid was poured out. Wash it three times with PBST. 100 μL Secondary antibody at a dilution ratio of 1:8000 (diluted in PBST) was added to each well. Seal the plate and incubate in a 37° C. incubator for 1 h.

HRP Chromogenic Reaction

After incubation of the secondary antibody for 1 hour, poured out the liquid in the wells, wash it three times with PBST. Add 100 μL TMB one-component coloring solution to each well (stored at 4° C. in the dark, pour appropriate amount of TMB color solution, and it can be used after reaching room temperature) and incubate at room temperature or 37° C. for 10-30 min or longer, until the color changed to the expected extent, then add 100 μM hydrochloric acid or sulfuric acid solution to stop the reaction, the color of reaction solution changed from blue to yellow.

The absorbance was measured at 450 nm within 30 min after the termination of the reaction.

Save and export the data, use the scatter plot to produce the standard curve, and obtain the standard curve formula to calculate the content of Ghrelin or acylated Ghrelin.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

SEQUENCE LISTING

| | |
|---|---|
| <110> | Wenzhou Kangning Hospital Co., Ltd |
| <120> | ADHD markers and their applications and kits |
| <130> | 2017 |
| <160> | 2 |
| <170> | PatentIn version 3.3 |
| <210> | 1 |
| <211> | 472 |
| <212> | DNA |
| <213> | Zebrafish (Barchydanio rerio var) |
| <400> | 1 |

| | |
|---|---|
| atgcctctga ggtgccgtgc cagcagcatg tttctgctcc tgtgtgtttc tctttccttg | 60 |
| tgtctcgagt ctgtgagcgg tggcaccagc ttcctcagtc cgactcagaa accgcagggt | 120 |
| cgaaggccac caagagtggg cagaagagaa gctgctgatc cagagatacc agtgatcaaa | 180 |
| gaagacgatc ggtttatgat gagcgctcca tttgaactgt ccatgtctct gagcgaagct | 240 |
| gaatatgaga aatatggtcc cgtgcttcag aatcttctgg aggatcttct tagagactct | 300 |
| tctttcgagt tctgacaaga gtcctacaaa gttcctcctt ataagcaatt gacaatattc | 360 |
| acaatttatt aatgatgtca tttatgggtt taacaaataa agaatgataa taaattattc | 420 |
| tctattctat gttctttatt ctgtagcaaa gtgggtgcat tgttacattg tt | 472 |

| | |
|---|---|
| <210> | 2 |
| <211> | 463 |
| <212> | DNA |
| <213> | Artificial sequence |
| <400> | 2 |

| | |
|---|---|
| atgcctctga ggtgccgtgc cagcagcatg tttctgctcc tgtgtgtttc tctttcctag | 60 |
| tctgtgagcg gtggcaccag cttcctcagt ccgactcaga aaccgcaggg tcgaaggcca | 120 |
| ccaagagtgg gcagaagaga agctgctgat ccagagatac cagtgatcaa agaagacgat | 180 |
| cggtttatga tgagcgctcc atttgaactg tccatgtctc tgagcgaagc tgaatatgag | 240 |
| aaatatggtc ccgtgcttca gaatcttctg gaggatcttc ttagagactc ttctttcgag | 300 |
| ttctgacaag agtcctacaa agttcctcct tataagcaat tgacaatatt cacaatttat | 360 |
| taatgatgtc atttatgggt ttaacaaata aagaatgata ataaattatt ctctattcta | 420 |
| tgttctttat tctgtagcaa agtgggtgca ttgttacatt gtt | 463 |

What is claimed is:

1. An attention deficit hyperactivity disorder (ADHD) marker, wherein the marker is ghrelin.

2. The ADHD marker according to claim 1, wherein human peripheral blood or cerebrospinal fluid is collected and an expression level of the ghrelin is detected to screen for potential ADHD patients.

3. An application of the ADHD marker according to claim 1 in the preparation of a reagent for detection of ADHD.

4. An application of a receptor of the ADHD marker according to claim 1 in the preparation of a drug for the treatment of ADHD, wherein a receptor of the ghrelin is used as a drug target for the treatment of ADHD.

5. An application of a receptor agonist of the ADHD marker according to claim 1 in the preparation of a drug for the treatment of ADHD.

6. The application according to claim 5, wherein the receptor agonist is methanesulfonic acid.

7. A kit for detecting ADHD, wherein the kit comprises a reagent for detecting ghrelin in peripheral blood or cerebrospinal fluid.


SEQUENCE LISTING (2 sequences)

SEQ ID NO: 1 - DNA, 472 bp, Barchydanio rerio var
atgcctctgaggtgccgtgccagcagcatgtttctgctcctgtgtgtttctctttccttgtgtctcgagtctgtgagcggtggcaccagcttcctcagtccgactcagaaaccgcagggtcgaaggccaccaagagtgggcagaagagaagctgctgatccagagataccagtgatcaaagaagacgatcggtttatgatgagcgctccatttgaactgtccatgtctctgagcgaagctgaatatgagaaatatggtcccgtgcttcagaatcttctggaggatcttcttagagactcttctttcgagttctgacaagagtcctacaaagttcctccttataagcaattgacaatattcacaatttattaatgatgtcatttatgggtttaacaaataaagaatgataataaattattctctattctatgttctttattctgtagcaaagtgggtgcattgttacattgtt

SEQ ID NO: 2 - DNA, 463 bp, Barchydanio rerio var
atgcctctgaggtgccgtgccagcagcatgtttctgctcctgtgtgtttctctttcctagtctgtgagcggtggcaccagcttcctcagtccgactcagaaaccgcagggtcgaaggccaccaagagtgggcagaagagaagctgctgatccagagataccagtgatcaaagaagacgatcggtttatgatgagcgctccatttgaactgtccatgtctctgagcgaagctgaatatgagaaatatggtcccgtgcttcagaatcttctggaggatcttcttagagactcttctttcgagttctgacaagagtcctacaaagttcctccttataagcaattgacaatattcacaatttattaatgatgtcatttatgggtttaacaaataaagaatgataataaattattctctattctatgttctttattctgtagcaaagtgggtgcattgttacattgtt